SEQUENCE LISTING (36 sequences)

SEQ ID NO: 1 - DNA, 22 bp, Artificial Sequence
  Description of Artificial Sequence Synthetic primer
tccccaatccttactttttgtc

SEQ ID NO: 2 - DNA, 18 bp, Artificial Sequence
  Description of Artificial Sequence Synthetic primer
cctcaatcctctgcggca

SEQ ID NO: 3 - DNA, 21 bp, Artificial Sequence
  Description of Artificial Sequence Synthetic primer
gcttcttgatgggtgatcaat

SEQ ID NO: 4 - DNA, 18 bp, Artificial Sequence
  Description of Artificial Sequence Synthetic primer
cctcaatcctctgcagcg

SEQ ID NO: 5 - DNA, 17 bp, Artificial Sequence
  Description of Artificial Sequence Synthetic primer
agcccggggcaagaggc

SEQ ID NO: 6 - DNA, 24 bp, Artificial Sequence
  Description of Artificial Sequence Synthetic primer
tttccagcttgtctgcaggaggag

SEQ ID NO: 7 - DNA, 32 bp, Artificial Sequence
  Description of Artificial Sequence Synthetic primer
ttttttttttttttacccggacctgtcsttgg

SEQ ID NO: 8 - DNA, 36 bp, Artificial Sequence
  Description of Artificial Sequence Synthetic primer
tttttttttttttttttgggctttccagagcagrga

SEQ ID NO: 9 - DNA, 42 bp, Artificial Sequence
  Description of Artificial Sequence Synthetic primer
ttttttttttttttttttttttctccgaaggtgaggtaacag

SEQ ID NO: 10 - DNA, 49 bp, Artificial Sequence
  Description of Artificial Sequence Synthetic primer
ttttttttttttttttttttttttttttccatagagaagagggaycaca

SEQ ID NO: 11 - DNA, 55 bp, Artificial Sequence
  Description of Artificial Sequence Synthetic primer
tttttttttttttttttttttttttttttttttttgctgcctcagctgcwtctcc

SEQ ID NO: 12 - DNA, 60 bp, Artificial Sequence
  Description of Artificial Sequence Synthetic primer
ttttttttttttttttttttttttttttttttttttttttccttgtgctgcctcagctgc

SEQ ID NO: 13 - DNA, 67 bp, Artificial Sequence
  Description of Artificial Sequence Synthetic primer
ttttttttttttttttttttttttttttttttttttttttttttttggacagctcctggaagggcac

SEQ ID NO: 14 - DNA, 74 bp, Artificial Sequence
  Description of Artificial Sequence Synthetic primer
tttttttttttttttttttttttttttttttttttttttttttttttcaccctctcctgggccgtggttttttt

SEQ ID NO: 15 - DNA, 81 bp, Artificial Sequence
  Description of Artificial Sequence Synthetic primer
ttttttttttttttttttttttttttttttttttttttttttttttttttttttttttttccccagattcagcagcgactg

SEQ ID NO: 16 - DNA, 87 bp, Artificial Sequence
  Description of Artificial Sequence Synthetic primer
ttttttttttttttttttttttttttttttttttttttttttttttttttttttttttttttttttatcgccgaggtgctgcgcctg

SEQ ID NO: 17 - DNA, 93 bp, Artificial Sequence
  Description of Artificial Sequence Synthetic primer
ttttttttttttttttttttttttttttttttttttttttttttttttttttttttttttttttttttttttctgcaggccttcacgctgctg

SEQ ID NO: 18 - DNA, 21 bp, Artificial Sequence
  Description of Artificial Sequence Synthetic primer
ccaatgagactggtgtcattc

SEQ ID NO: 19 - DNA, 20 bp, Artificial Sequence
  Description of Artificial Sequence Synthetic primer
cagcatagcaagaacccatc

SEQ ID NO: 20 - DNA, 21 bp, Artificial Sequence
  Description of Artificial Sequence Synthetic primer
ccaagaggaccattgaggaag

SEQ ID NO: 21 - DNA, 21 bp, Artificial Sequence
  Description of Artificial Sequence Synthetic primer
gctgtggagaaacagtgtgag

SEQ ID NO: 22 - DNA, 20 bp, Artificial Sequence
  Description of Artificial Sequence Synthetic primer
ggaaagcccacaagaagctc

SEQ ID NO: 23 - DNA, 20 bp, Artificial Sequence
  Description of Artificial Sequence Synthetic primer
agcatgagaatgcagctgtg

SEQ ID NO: 24 - DNA, 20 bp, Artificial Sequence
  Description of Artificial Sequence Synthetic primer
gaaggagccttttgcttgtc

SEQ ID NO: 25 - DNA, 21 bp, Artificial Sequence
  Description of Artificial Sequence Synthetic primer
cactgagaccacagcaaacac

SEQ ID NO: 26 - DNA, 19 bp, Artificial Sequence
  Description of Artificial Sequence Synthetic primer
cctccaccacattttcacg

SEQ ID NO: 27 - DNA, 20 bp, Artificial Sequence
  Description of Artificial Sequence Synthetic primer
cacagtcatcattccgaacc

SEQ ID NO: 28 - DNA, 20 bp, Artificial Sequence
  Description of Artificial Sequence Synthetic primer
gaccaagaaactttcgctcc

SEQ ID NO: 29 - DNA, 20 bp, Artificial Sequence
  Description of Artificial Sequence Synthetic primer
tgtaaacacagtgctgcgag

SEQ ID NO: 30 - DNA, 18 bp, Artificial Sequence
  Description of Artificial Sequence Synthetic primer
caccctctgcaggagagc

SEQ ID NO: 31 - DNA, 19 bp, Artificial Sequence
  Description of Artificial Sequence Synthetic primer
cttgctcaatgccaccatc

SEQ ID NO: 32 - DNA, 20 bp, Artificial Sequence
  Description of Artificial Sequence Synthetic primer
taaaaacctggagccactgg

SEQ ID NO: 33 - DNA, 21 bp, Artificial Sequence
  Description of Artificial Sequence Synthetic primer
tgaccatccctctcaatcttc

SEQ ID NO: 34 - DNA, 21 bp, Artificial Sequence
  Description of Artificial Sequence Synthetic primer
tccctctttcctgccactcct

SEQ ID NO: 35 - DNA, 20 bp, Artificial Sequence
  Description of Artificial Sequence Synthetic primer
acaggctctggccaattctc

SEQ ID NO: 36 - DNA, 20 bp, Artificial Sequence
  Description of Artificial Sequence Synthetic primer
acctggccgtggctctgttc

(19) United States
(12) Patent Application Publication
(10) Pub. No.: US 2006/0029930 A1
(43) Pub. Date: Feb. 9, 2006

(54) Detecting genotypes associated with congenital adrenal hyperplasia

(75) Inventor: Jennifer Dianne Keen-Kim, Playa Vista, CA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 10/914,642

(22) Filed: Aug. 9, 2004

(51) Int. Cl. C12Q 1/70; C12Q 1/68; C12P 19/34

(52) U.S. Cl. 435/5; 435/6; 435/91.2

(57) ABSTRACT

The present invention provides methods of determining the genotype at a duplicated region of a genome. The methods involve (a) amplifying the DNA, preferably using four amplification primers. The exemplary methods produce a first amplicon corresponding to a first distinct region or gene by utilizing a first primer and a second primer which are selected to produce the first amplicon in the presence of the first distinct region or gene, but not in absence of the first distinct region or gene. A second amplicon corresponding to a second distinct region or gene is also produced by utilizing a third primer and a fourth primer which are selected to produce the second amplicon in the presence of the second distinct region or gene, but not in the absence of the second distinct region or gene. A third and/or fourth amplicon corresponding to a hybrid gene or genes is/are also produced if a hybrid gene is present.

### CROSS-REFERENCE TO RELATED APPLICATIONS

This application is related to U.S. application Ser. No. 10/417,804 filed Apr. 16, 2003, the entire contents of which are hereby incorporated by reference.

### FIELD OF INVENTION

The present invention involves the determination of genotypes in congenital adrenal hyperplasia.

### BACKGROUND OF THE INVENTION

The following discussion of the background of the invention is merely provided to aid the reader in understanding the invention and is not admitted to describe or constitute prior art to the present invention.

Congenital Adrenal Hyperplasia (CAH) is characterized by a defect in adrenal steroid biosynthesis causing reduced corticoid production and increased androgen production. CAH manifests in a variety of phenotypic severities, which are broadly classified as classic and non-classic disease. Classical disease is more severe, and includes phenotypes such as ambiguous genitalia in females in utero (classic virilization), adrenal crisis, and salt wasting. Non-classic disease includes less severe phenotypes including virilization in childhood, and in women, hirsutism, inconsistent menstruation, and infertility. See, e.g., The Merck Manual of Diagnosis and Therapy, 17^{th} ed., Beers and Berkow, Eds. (1999), pages 2381-82.

Approximately 90% of CAH is caused by 21-hydroxylase deficiency attributable to mutations in the gene coding for 21-hydroxylase, also referred to as CYP21A2. The 21-hydroxylase gene lies on chromosome 6p21.3 among genes that code for proteins determining human leukocyte antigen (HLA) types. The 21-hydroxylase gene locus also has a pseudogene called CYP21A, which is about 30 kilobases (kb) away from CYP21A2. CYP21A is approximately 98% homologous in structure to CYP21A2, but is rendered inactive due to minor differences in the gene as compared to the active CYP21A gene. The proximity and homology of CYP21A2 with CYP21A is thought to predispose the CYP21A2 to crossovers in meiosis between CYP21A2 and CYP21A, which can result in the loss of gene function.

A number of point mutations and small deletions have been identified that deactivate this second copy. These differences include (in order from exons 1-10): P30L, IVS2-13 A/C>G, Δ8 bp, I172N, exon 6 cluster mutation (I235N, V236E, and M238K), V281L, F306+t, Q318X, and R356W. Transfer of any of these mutations from the pseudogene CYP21A to the functional CYP21A2 can occur through gene conversion or non-homologous recombination, thus inactivating the functional CYP21A2. Another source of mutation at the CYP21 locus is genomic rearrangement resulting in duplication or deletion of entire segments of the gene region. Large 30 kb deletions that fuse CYP21A with CYP21A2 account for approximately 25% of 21-hydroxylase deficiency. These types of mutation at the CYP21 gene locus account for approximately 90% of 21-hydroxylase deficiency. The remaining 10% of 21-hydroxylase deficiency is caused by rare sporadic mutation. The fact that this locus involves an evolutionary gene duplication produces technical difficulties for diagnosing mutation in the functional CYP21A2. Wedell & Luthman, Hum Genet (1993) 91:236-240; Speiser et al., Molecular Genetics and Metabolism, 71: 527-534 (2000); For a review see White & Speiser, Endocrine Reviews 21(3): 245-291 (2000).

The core problem in CAH is the inability of the adrenal glands to make enough cortisol in the non-salt wasting form, or enough cortisol and salt-retaining hormone in the salt-wasting form. Instead of making cortisol, the hormonal raw materials which usually make cortisol are shifted away to make other hormones, specifically male sex hormones (androgens). As a result, more androgens are produced than necessary. Before birth, the excess androgens stimulate the growth of the genitalia. When the child is male this does not cause grave problems. However excess androgens in a female with this disorder causes the child's genitalia to have the appearance of a male although the internal genitalia are normal female. Therefore, methods of identifying the genotype of an individual at the CYP21A2 gene would result in early diagnosis and treatment of this disease.

CAH is an example of a disease associated with a duplicated gene; that is, a gene that is represented in multiple copies within a genome. In particular, CAH sequences in the human genome comprise both a “functional” gene that encodes an enzymatically active 21-hydroxylase protein, and a “pseudogene” that contains the inactive gene. Numerous genes are duplicated in the genome of both eukaryotes and prokaryotes. In humans, a number of diseases are associated with recombination between duplicated gene sequences, including Gaucher disease, familial juvenile nephronophthisis, fascioscapulohumeral muscular dystrophy, spinal muscular atrophy, polycystic kidney disease, chronic granulomatous disease, Hunter syndrome, Charcot-Marie Tooth disease (CMT1A)/hereditary neuropathy with liability to pressure palsies, neurofibromatosis, ichthyosis, red-green color blindness, hemophilia A, incontinentia pigmenti, Emery Dreifuss muscular dystrophy, Shwachman-Diamond Syndrome, Williams-Beuren syndrome, Angelman syndrome/15q11.2-q13 Prader-Willi syndrome, DiGeorge/velocardiofacial syndrome, debrisoquine resistance, Chr 22-BL, Chr 22HsPOX2/DGR6 schizophrenia, Smith-Magenis syndrome, Dup 17p11.2 syndrome, cat eye syndrome, Chr 22-BE1, Chr 22-BE2, and Chr 22-BK.

### SUMMARY OF THE INVENTION

The present invention provides methods and compositions useful for determining the genotype of a duplicated region of a genome, also referred to hereinafter as “duplicated genes” in which the distinct gene sequences of the duplication are predisposed to recombination. The duplicated gene can comprise both a functional gene and a pseudogene.

The methods are applicable for determining the genotype for 21-hydroxylase (CYP21) locus that includes a pseudogene (CYP21A) and a related functional gene (CYP21A2). The methods involve amplifying genomic DNA to produce one or more amplicons from the CYP21 locus. By careful selection of amplification primers, four primers may be used to generate the following amplicons (i) a first amplicon corresponding to an intact CYP21A pseudogene, if present, utilizing a first primer and a second primer that produce the first amplicon in the presence of the intact pseudogene but not in the absence of the intact pseudogene; (ii) a second amplicon corresponding to an intact CYP21A2 gene, if present, by utilizing a third primer and a fourth primer that produces the second amplicon in the presence of the intact CYP21A2 gene but not in absence of the intact CYP21A2 gene. Two of the primers also amplify a CYP21A/CYP21A2 fusion gene if present to produce a third amplicon and two of the primers amplify a CYP21A2/CYP21A rearranged gene if present to produce a fourth amplicon. A schematic description of exemplary primers for use in this method is provided in FIG. 1.

The skilled artisan will understand that, for each allele of the CYP21 gene locus, the presence of the fusion gene can occur in the presence or absence of the intact linked CYP21A pseudogene, intact linked CYP21A2 gene, and the linked CYP21A2/CYP21A rearranged gene; likewise the presence of the rearranged gene can occur in the presence or absence of the intact CYP21A linked pseudogene, intact linked CYP21A2 gene, and the linked CYP21A/CYP21A2 fusion gene; and the intact pseudogene or active gene can occur in the presence or absence of the linked CYP21A/CYP21A2 fusion gene and the linked CYP21A2/CYP21A rearranged gene. The four amplification primers described herein, and their equivalents, can be used to identify the presence or absence of the fusion gene, the rearranged gene, the intact pseudogene, and the intact active gene.

A “hybrid” gene refers to a genetically rearranged gene resulting in the whole or partial fusion of two distinct genes. The term “fusion gene” as used herein refers to a hybrid gene created by joining portions of two different genes, e.g., the CYP21A2 and CYP21A genes, without altering the relative 3′-to-5′ order of the fused sequences. A fusion gene can result from the deletion of a nucleic acid sequence in between two genes, thus causing the two genes to become “fused” without altering the 3′-to-5′ order of the sequences remaining in the fused gene. In the present invention a nucleic acid sequence in between the CYP21A2 and CYP21A genes is sometimes deleted, causing the formation of a fusion gene, which contains parts of the CYP21A2 and CYP21A genes. FIG. 1 depicts an example of a fusion gene. Sequences upstream from the CYP21A gene can remain upstream of the fusion gene, and sequences downstream from the CYP21A2 gene can remain downstream of the fusion gene, providing binding sequences for the primers of the present invention.

The term “rearranged gene” as used herein refers to a hybrid gene created by joining portions of two different genes, e.g., the CYP21A2 and CYP21A genes, in which the relative 3′-to-5′ order of the fused sequences is altered. In the present invention, the “first half” of the CYP21A2 gene can become fused to the “second half” of the CYP21A gene, causing the formation of a rearranged gene, which contains parts of the CYP21A2 and CYP21A genes. FIG. 1 depicts an example of a rearranged gene. Sequences upstream from the CYP21A gene can remain upstream of the rearranged gene, and sequences downstream from the CYP21A gene can remain downstream of the rearranged gene, providing binding sequences for the primers of the present invention.

The skilled artisan will understand that duplicated regions of the genome are very dynamic. For example, hybrid genes can also arise by gene conversion (e.g., genetic change without exchange of flanking markers). Thus, hybrid genes may arise with or without the concomitant loss of a “parent” duplicated gene sequence. See, e.g., Chung et al., Am. J. Hum. Genet. 71(4):823-37 (2002).

The sample analyzed in the present methods is a sample of DNA, preferably a sample of genomic DNA. By “genomic DNA” is meant a sample of DNA from an organism that contains coding and noncoding (intron and other) sequences of DNA of the organism. For example, a sample of genomic DNA may be obtained from a blood sample by isolating the DNA from nucleated cells in the sample.

Any sample comprising genomic DNA may be used as the source of material to be genotyped. The term “biological sample” as used herein refers to a sample obtained from a biological source, e.g., an organism, cell culture, tissue sample, etc. A biological sample can, by way of non-limiting example, consist of or comprise blood, sera, urine, feces, epidermal sample, skin sample, cheek swab, sperm, amniotic fluid, cultured cells, bone marrow sample and/or chorionic villi. Biological samples may be obtained from plants, animals, fungi, etc.

The term “subject” as used herein refers to any eukaryotic organism. Preferred subjects are fungi, plants, invertebrates, insects, arachnids, fish, amphibians, reptiles, birds, marsupials and mammals. A mammal can be a cat, dog, cow, pig, horse, ox, elephant, simian. Most preferred subjects are humans. A subject can be a patient, which refers to a human presenting to a medical provider for diagnosis or treatment of a disease. Methods to study the role of cytochrome P450 enzymes in metabolism in animals are known (see, e.g., Chauret et al., In Vitro Comparison of P450-Mediated Metabolic Activities in Human, Dog, Cat, and Horse, Drug Metabolism and Disposition 25:1130-1136, 1997). The term “animals” includes metamorphic and prenatal forms of animals.

In the disclosure, a “plurality of samples” refers to at least two. Preferably, a plurality refers to a relatively large number of samples. A plurality of samples is from about 5 to about 500 samples, preferably about 25 to about 200 samples, most preferably from about 50 to about 200 samples. Samples that are processed in a single batch run of the method of the invention are usually prepared in plates having 24, 48, 96, 144, or 192 wells. The term “samples” includes samples per se as well as controls, standards, etc. that are included in a batch run.

The term “amplifying” as used herein refers to an increase in the copy number of nucleic acid sequences. Nucleic acid sequences can be amplified as necessary for further use using amplification methods, such as PCR, isothermal methods, rolling circle methods, etc., well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. Jun. 1, 2001;29(11):E54-E54; Hafner et al., Biotechniques April 2001; 30(4):852-6, 858, 860 passim; Zhong et al., Biotechniques April 2001;30(4):852-6, 858, 860 passim. Amplification is preferably performed by the polymerase chain reaction (PCR). The “polymerase chain reaction” (or PCR amplification) is a method for copying and amplifying specific sequences of nucleotides in DNA using a heat-stable polymerase and two nucleic acid primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. PCR also can be used to detect the existence of the defined sequence in a DNA sample.

The term “amplicon” as used herein refers to a product of an amplification reaction that is a copy of a particular DNA sequence.

In some embodiments a control amplicon is also generated as a control for the PCR reaction. Typically, a control amplicon may be generated from a sequence known to be present in the DNA sample. Such sequences are often “housekeeping” genes, but may be any genomic sequence that is unrelated to the genomic sequence of interest. The control amplicon is generated in the absence of any of the test amplicons to identify false negatives; that is, when an amplicon is absent, it may be due to either the absence of the target sequence, or may be due to a failure of the entire amplification reaction. A control amplicon for use with the methods disclosed herein can be from the AVPR2 gene.

Nucleic acid “primers” are oligonucleotides that bind to and direct polymerization activity at a particular region of a nucleic acid molecule. In PCR, primers define the area of the genome to be amplified by binding to it and directing the activity of DNA polymerase to that sequence.

The term “distinctively labeled” as used herein refers to each member of a set that is labeled with a distinct label so that each member can be distinguished from the other members. For example, in a set of distinctively labeled nucleotides (e.g., dideoxy NTPs, or ddNPTs), each type of “N” (nucleotide) is labeled with a label that can be distinguished from the other types of labels. Thus, for example, if four labels designated 1, 2, 3, and 4 are used to label the four types of ddNTPs, each ddATP molecule is labeled with label “*1”, each ddTTP molecule is labeled with label “*2”, each ddCTP molecule is labeled with label “*3”, and each ddGTP molecule is labeled with label “*4”. In some embodiments of the invention, the distinctive label is a fluorescent label. In preferred embodiments the signal is provided by each ddNTP having a different color.

Amplicons prepared as described are preferably used for the subsequent analysis of one or more predetermined single nucleotide polymorphisms (“SNPs”). In preferred embodiments, this analysis comprises contacting one or more of the amplicon(s) produced with one or more extension primers selected to indicate the presence of a predetermined single nucleotide polymorphism under conditions where the extension primers are extended by the addition of a distinctively labeled ddNTP from a set of ddNTPs. The identity of the distinctively labeled ddNTP added to each of the extended extension primer(s) can be used to determine the presence or absence of one or more CYP21A2 SNPs. Alternative methods for analyzing SNPs include flow cytometric methods; sequencing methods; array hybridization; mismatch detection; restriction/digestion, mass spectrometry methods, etc. See, e.g., Taylor et al., Biotechniques 30:661-6, 668-9 (2001); Landegren et al., Genome Research 8: 769-76 (1998); and U.S. Pat. No. 6,043,031; each of which is hereby incorporated in their entirety.

In various embodiments each of the extension primers used in the present methods is designed to have a different molecular weight, and the extension primers are extended by the addition of one nucleotide through a single base extension reaction. The different molecular weights may be generated by the presence of one or more of nucleotides attached to the extension primers that do not hybridize to the CYP21 gene locus. For example, a stretch of poly-T, poly-A, poly-C, or poly-G may be added to each extension primer in the extension primer set to generate different molecular weights. In this manner, each extension primer may be identified by its size, and hence its migration distance or rate in a separation system (e.g., a capillary electrophoresis system).

In a particularly preferred embodiment the ddNTPs contain a detectable label, most preferably a fluorescent label, and the set of ddNTPs contains ddATP, ddCTP, ddGTP, and ddTTP, each of which is labeled with a distinct detectable label. Preferred detectable labels are described hereinafter.

In preferred embodiments, the amplification primers are selected so that first amplicon is about 4.0 kb in length, corresponding to the CYP21A pseudogene; the second amplicon is about 3.4 kb in length corresponding to the CYP21A2 functional gene; the third amplicon is about 4.0 kb in length corresponding to the CYP21A/A2 fusion gene; and the fourth amplicon is about 3.4 kb in length corresponding to the CYP21A2/A rearrangement gene. In the most preferred embodiment the first and second primers flank the CYP21A pseudogene if present; and the third and fourth primers flank the CYP21A2 active gene if present; the first and fourth primers flank the CYP21A/CYP21A2 fusion gene if present; and the third and second primers flank the CYP21A2/CYP21A rearranged gene if present. The term “about” as used herein means ±5%. These embodiments are exemplary only, and the skilled artisan will understand that numerous primers could be selected to provide a variety of differently sized amplicons.

In particularly preferred embodiments, the primers selected have one or more of the following sequences: the first primer has the sequence SEQ ID NO 1: 5′-TCCCCAATCCTTACTTTTTGTC-3′; the second primer has the sequence SEQ ID NO 2: 5′-CCTCAATCCTCTGCGGCA-3′; the third primer has the sequence SEQ ID NO 3: 5′-GCTTCTTGATGGGTGATCAAT-3′; and the fourth primer has the sequence SEQ ID NO 4: 5′-CCTCAATCCTCTGCAGCG′-3′. The skilled artisan will understand that complementary or conservative sequences thereof may also be used.

A “complementary sequence” as used herein refers the opposing strand of a nucleic acid duplex, corresponding to Watson-Crick base pairing rules. Thus, a strand having an A, T, G, and C in particular locations on the strand would have T, A, C, and G on the complementary strand. Conservative sequences are sequences having at least 60% homology to the sequence of interest, in this case a nucleic acid primer. In other embodiments conservative sequences have at least 70% homology or at least 75% homology or at least 80% homology at least 85% homology at least 90% homology or at least 95% homology or at least 98% homology. For purposes of the present invention, the degree of homology between two nucleic acid sequences is determined by using GAP version 8 from the GCG package with standard penalties for DNA: GAP weight 5.00, length weight 0.300, Matrix described in Gribskov and Burgess, Nucl. Acids Res. 14(16); 6745-6763 (1986).

In preferred embodiments, the determination of which amplicons are produced is determined by agarose gel electrophoresis, for example on a 0.7% to 1% agarose slab gel. In still other preferred embodiments, the determination of which distinctively labeled ddNTP is present in each of the extended extension primer(s) is determined by capillary electrophoresis. In particularly preferred embodiments, capillary electrophoresis is coupled with fluorescence detection to detects the distinctively labeled ddNTP present.

In various embodiments, the extension primers used bind to one or more sequences characteristic of a genotype selected from: IVS2-13 A/C>G, 1172N, V281L, Q318X, R356W, I235N, V236E, M238K, F306+t, Δ8 bp-R, P30L, Δ8 bp-F, and P453S.

As used herein, “primer extension” refers to the enzymatic extension of the three-prime (3′) hydroxy group of an extension primer, which is an oligonucleotide X nucleotides long that is paired to a template nucleic acid (for an example of primer extension as applied to the detection of polymorphisms, see Fahy et al., Multiplex fluorescence-based primer extension method for quantitative mutation analysis of mitochondrial DNA and its diagnostic application for Alzheimer's disease, Nucleic Acid Research 25:3102-3109, 1997). The extension reaction is catalyzed by a DNA polymerase. Primer extension reactions can be “single base extensions” where the primer is extended by a single base. By “DNA Polymerase” is meant a DNA polymerase, or a fragment thereof, that is capable of carrying out primer extension. Thus, a DNA polymerase can be an intact DNA polymerase, a mutant DNA polymerase, an active fragment from a DNA polymerase, such as the Klenow fragment of E. coli DNA polymerase, and a DNA polymerase from any species including, but not limited to, thermophiles.

Extension of the 3′ end of the oligonucleotide generates an oligonucleotide having a length of at least (X+Y) nucleotides, where Y≧one, having a sequence that is the reverse complement of the template nucleic acid. If one of the nucleotides in the added sequence Y is labeled, then the extended (X+Y) oligonucleotide is labeled. Four different ddNTPs, each distinctively labeled, can be present as the only nucleotides in the reaction mixture so that when a single base is added by extension, the identity of the base can be revealed by its distinctive label. The use of only di-deoxy nucleotides in such a reaction ensures that only a single base is added during extension.

In preferred embodiments, each extension primer has a nucleotide sequence that binds in a complementary fashion to a portion of a sequence of nucleic acid that represents the genotype at the CYP21 gene. Preferred extension primers are of a length sufficient to provide specific binding to the sequence of interest. Such primers comprise an exact complement to the sequence of interest for 10 to 40 nucleotides in length, and preferably 20 to 30 nucleotides in length. The extension primer sequence has a 3′ terminus that pairs with a nucleotide base that is, in the sample nucleic acid to which the primer is hybridized, 5′ from the site of one or more bases in the sequence of interest that represent a polymorphism in a gene. In addition to this sequence the primer in the preferred embodiments has a polymer “tail,” or portion that distinguishes the primer based on molecular weight. The polymer is preferably a poly-thymine tail from 2 or 3 nucleotides in length up to any individual number of nucleotides, such as about 50 or 75 nucleotides or 75-100 nucleotides (or 50-75 nucleotides or 75-100 nucleotides or greater than 100 nucleotides) in length or even a greater number of nucleotides. Exemplary extension primers for use in CYP21 genotyping are disclosed, e.g., in Krone et al., Clin. Chem. 48(6) 818-825 (2002).

An amount of nucleic acid sufficient for primer extension can, but need not be, prepared by amplification via polymerase chain reaction (PCR) using PCR primers. As a non-limiting example, when the preselected CAH gene is CYP21A, an appropriate PCR primer includes, but is not limited to, one or more, two or more, three or more, four or more, five or more, six or more, seven or more, eight or more, nine or more, ten or more, eleven or more, twelve or more, and preferably all, of those having sequences selected from the group provided in table 1 below:

TABLE 1
Primers for Primer Extension

| Mutation | SEQ ID | |
|---|---|---|
| locus | NO: | Sequence |
| P30L | 5 | 5′-AGCCCGGGGCAAGAGGC-3′ |
| In2G | 6 | 5′-T₃CCAGCTTGTCTGCAGGAGGAG-3′ |
| Δ8bp-F | 7 | 5′-T₁₄ACCCGGACCTGTCSTTGG-3′ |
| Δ8bp-R | 8 | 5′-T₁₇GGGCTTTCCAGAGCAGRGA-3′ |
| I172N | 9 | 5′-T₂₂CTCCGAAGGTGAGGTAACAG-3′ |
| 1235N | 10 | 5′-T₂₈CCATAGAGAAGAGGGAYCACA-3′ |
| V236E | 11 | 5′-T₃₅GCTGCCTCAGCTGCWTCTCC-3′ |
| M238K | 12 | 5′-T₄₀CCTTGTGCTGCCTCAGCTGC-3′ |
| V28IL | 13 | 5′-T₄₆GGACAGCTCCTGGAAGGGCAC-3′ |
| F306 + T | 14 | 5′-T₄₇CACCCTCTCCTGGGCCGTGGTTTTTTT-3′ |
| Q318X | 15 | 5′-T₆₀CCCCAGATTCAGCAGCGACTG-3′ |
| R356W | 16 | 5′-T₆₆ATCGCCGAGGTGCTGCGCCTG-3′ |
| P453S | 17 | 5′-T₇₂CTGCAGGCCTTCACGCTGCTG-3′ |

In the above table, the designation of Tₙ wherein n equals the number indicated means a run of Ts equal to the number indicated. Thus, T₃ as in SEQ ID NO:6 refers to a sequence of three Ts (i.e., TTT).

For each reaction mixture, the amount of the nucleic acid sufficient for primer extension or for performing PCR amplification is determined by obtaining a sample comprising nucleic acid and determining the concentration of nucleic acid therein. One skilled in the art will be able to prepare such samples to a concentration and purity necessary to practice the invention, and to estimate the amount of a specific sample that should be added to a particular reaction mixture. A failure to detect a signal in the method of the invention signifies that, among other things, an inadequate amount of nucleic acid has been added to a reaction mixture. Those skilled in the art will be able to trouble-shoot failed batch runs and adjust the contents of the reaction mixtures and/or conditions of the run accordingly. Control samples can be included in the batch runs to confirm that appropriate amounts of nucleic acid are present.

One or more of steps of the foregoing methods, or combinations thereof, are preferably performed automatically, typically using robotics, in order to provide for the processing of a large number of samples in a single batch run. Preferred forms of automation will provide for the preparation and separation of a plurality of labeled nucleic acids in small volumes. The term “small volumes” refers to volumes of liquids less than 2 ml, e.g., any volume from about 0.001 picoliters or about 0.001 μl, to any volume of about 2 ml, 500 μl, 200 μl, 100 μl, 10 μl, 1 μl, 0.1, 0.2 μl, μl, 0.01 μl, or 0.001 μl.

After primer extension, the set of distinctively labeled polynucleotides can be separated from each other so that each is mobilized in a manner that relates to each of their specific positions in the respective nucleotide sequence, and the detection of the distinctive signals generated from the distinctively labeled polynucleotides occurs during or after the mobilization step. The distinctively labeled polynucleotides can be separated from each other by electrophoresis. A preferred form of electrophoresis is capillary electrophoresis, or any form of electrophoresis that allows for the separation of a plurality of labeled nucleic acids in small volumes by automated or semi-automated methods and devices.

The CYP21 mutations or variants can be of any type, including, but not limited to, deletions, inversions, insertions, translocations, mutations resulting in aberrant RNA splicing, single nucleotide polymorphisms, and combinations thereof. The CYP21 mutations include, but are not limited to, those reflected in Table 1.

In another aspect the present invention provides a substantially purified nucleic acid sample containing one or more, two or more, three or more, four or more, five or more, six or more, seven or more, eight or more, nine or more, ten or more, eleven or more, twelve or more, and preferably each of the nucleic acids selected from the group shown in Table 1, or a complementary or conservative nucleic acid sequence thereof. While the above primers pertain specifically to CAH, the person of ordinary skill will realize, with reference to the present disclosure, that primers specific for genotypes and mutations specific for other genetic diseases can also be designed using the same principles.

In preferred embodiments, one or more of the sequences are present at a concentration of about 100 nM, but the person or ordinary skill will realize that lower or higher concentrations of primers will also serve to accomplish PCR, such as concentrations of 150 nM, or more, 50 nM, 25 nM, 10 nM and even less. Furthermore, specific conditions of PCR can be utilized (e.g., longer cycling times) that will enable the usage of lower concentrations. A nucleic acid sample is amplified when the number of copies is increased by at least 1,000 times. More preferably the number of copies is increased by at least 10,000 times or at least 100,000 times or at least 1,000,000 times.

For CYP21A2 mutations not detectable using primer extension including but not limited to L433P, R483+C R408C and W407X/null, the complete CYP21A2 encoding gene can be cycle-sequenced in forward and reverse directions using the following primers:

TABLE 2

Primers for Sequencing CYP21A2

| | SEQ | |
|---|---|---|
| | ID | |
| Location | NO: | Sequence |
| 5′ untranslated | 18 | 5′-CCAATGAGACTGGTGTCATTC-3′ |
| region, forward | | |
| intron 2, reverse | 19 | 5′-CAGCATAGCAAGAACCCATC-3′ |
| exon 2, forward | 20 | 5′-CCAAGAGGACCATTGAGGAAG-3 |
| intron 3, reverse | 21 | 5′-GCTGTGGAGAAACAGTGTGAG-3′ |
| exon 3, forward | 22 | 5′-GGAAAGCCCACAAGAAGCTC-3′ |
| intron 5, reverse | 23 | 5′-AGCATGAGAATGCAGCTGTG-3′ |
| intron 7, reverse | 24 | 5′-GAAGGAGCCTTTTGCTTGTC-3′ |
| intron 7, forward | 25 | 5′-CACTGAGACCACAGCAAACAC-3′ |
| intron 9, reverse | 26 | 5′-CCTCCACCACATTTTCACG-3′ |
| exon 9, forward | 27 | 5′-CACAGTCATCATTCCGAACC-3′ |
| exon 10, reverse | 28 | 5′-GACCAAGAAACTTTCGCTCC-3′ |
| exon 10, forward | 29 | 5′-TGTAAACACAGTGCTGCGAG-3′ |
| intron 6, forward | 30 | 5′-CACCCTCTGCAGGAGAGC-3′ |
| exon 8, forward | 31 | 5′-CTTGCTCAATGCCACCATC-3′ |
| exon 5, forward | 32 | 5′-TAAAAACCTGGAGCCACTGG-3′ |

The skilled artisan will understand that complementary or conservative sequences thereof of SEQ ID NO: 18-32 may also be used. Also included are combinations of these primers in an aqueous medium including, but not limited to, one or more, two or more, three or more, four or more, five or more, six or more, seven or more, eight or more, nine or more, ten or more, eleven or more, twelve or more, thirteen or more, fourteen or more and preferably all, of the primers.

In another aspect the present invention provides a kit containing one or more nucleic acids or sets of nucleic acids as described above in an amount sufficient to perform a polymerase chain reaction amplification of a nucleic acid sample and/or a single base extension reaction on a nucleic acid sample and/or cycle sequencing. The kits optionally contain reagents for performing a PCR amplification and/or a single base extension reaction, wherein the nucleic acids and reagents, if present, are provided in a container. The container is preferably a box or wrapping that encloses the items of the kit. In one embodiment the kit contains one or more nucleic acids of SEQ ID NOS. 5-17. In another embodiment the kit contains two or more nucleic acids of SEQ ID NOS. 18-32. The person of ordinary skill will realize with reference to the present disclosure that kits specific for the detection of other genetic diseases, or for a battery of genetic diseases, can be designed using the same principles.

In various aspects of the present invention, the genotyping methods described herein may be used to identify subjects at risk for diseases related to congenital adrenal hyperplasia. These methods may also be applied to determine carrier status (heterozygosity). The skilled artisan will understand that such methods may be applied to samples from individuals and for prenatal determinations.

In various other aspects, the invention provides a method for selecting a treatment regimen for a particular subject, based upon the identified 21-hydroxylase (CYP21) genotype of the subject. The methods comprise determining a genotype according to the foregoing methods, and selecting a treatment regimen known in the art to ameliorate one or more effects of the genotype identified. A “treatment regimen” is a course of treatment that may include, but is not limited to, drug therapy, changes to lifestyle, changes to diet, surgical intervention, etc. Methods for treatment of 21-hydroxylase deficiency are well known in the art. For example, 21-hydroxylase deficiency is the most frequent cause of ambiguous genitalia in the newborn female. The concordance between genotype and phenotype is sufficiently robust as to be relevant and useful in planning treatment strategies. Hughes, Semin. Reprod. Med. 20(3):229-42 (2002). The dose of glucocorticoid replacement in the early years of life can be tailored according to the predicted degree of 21-hydroxylase enzyme deficiency in the anticipation that this may avoid hitherto excessive steroid replacement during the critical early years of growth and development. The means to prevent genital virilization in affected females is clearly demonstrated by the success of early dexamethasone administration to pregnant mothers at risk. Moreover, individuals with salt losing 21-hydroxylase enzyme deficiency need to replace the lack of aldosterone, typically using Fludrocortisone in a dose of 50-300 micrograms. The correct level of fludrocortisone is determined by measuring blood pressure, potassium and the salt sensitive hormone renin in the blood.

In yet other preferred embodiments, the invention provides a method for selecting one or more subjects for inclusion in a clinical trial, based upon the identified 21-hydroxylase (CYP21) genotype of the subject(s). The methods comprise determining a genotype according to the foregoing methods, and selecting those patients to be included or excluded from the trial based, at least in part, on the genotype identified. In these embodiments, subjects may be excluded or included from the trial, according to their heightened risk of suffering from a disease and/or their predicted responsiveness to a particular treatment regimen. The person of ordinary skill will realize with reference to the present disclosure that treatment regimens specific for treating other genetic diseases can be designed using the same principles.

The summary of the invention described above is non-limiting and other features and advantages of the invention will be apparent from the following detailed description of the invention, and from the claims.

### BRIEF DESCRIPTION OF THE FIGURES

FIG. 1 provides a schematic illustration of the CYP21 gene area. Amplification primers are depicted as arrows, indicating the direction in which each primer initiates DNA polymerization.

FIG. 2 provides a schematic illustration of the CYP21A and CYP21A2 gene areas. EcoRI restriction enzyme cleavage sites are labeled with “RI”.

FIG. 3 is a schematic showing the identity of various mutations detected by primer extension minisequencing in the CYP21A2 gene.

FIG. 4 depicts minisequencing data and reporting scheme for wild-type alleles in the CYP21A2 amplicon. Upper result shows the scanning results in black and white (peaks are colored as described in Table 3). The reporting scheme for minisequencing shows different rows representing the four possible amplicons from the four PCR reactions (no: 1: CYP21A2; no:2: pseudogene; no.3: CYP21A/A2 and no. 4: CYP21A2/A) and the rows show the presence or absence of each the genotype of each locus based on minsequencing. Squares are colored solid black for wild-type alleles and gray shaded for mutant alleles, while heterozygous are stripped (none shown).

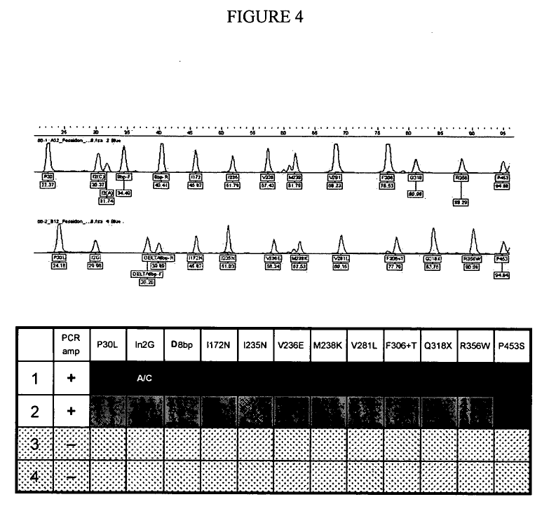

### DETAILED DESCRIPTION OF THE INVENTION

Congenital Adrenal Hyperplasia (CAH) is a genetic disease involving genome instability caused by genetic duplication. It is characterized by a defect in adrenal steroid biosynthesis causing reduced corticoid production and increased androgen production. CAH manifests in a variety of phenotypic severities, which are broadly classified as Classic and Non-classic disease. Classical disease is more severe, and includes phenotypes such as ambiguous genitalia/sex determination in females in utero (classic virilization), adrenal crisis, and salt wasting. Non-classic disease includes less severe phenotypes including virilization in childhood, and in women, hirsutism, inconsistent menstruation, and infertility. See, e.g., The Merck Manual of Diagnosis and Therapy, 17^{th} ed., Beers and Berkow, Eds. (1999), pages 2381-82.

Approximately 90% of CAH is caused by 21-hydroxylase deficiency attributable to mutations in the gene coding for 21-hydroxylase, also referred to as CYP21A2. The 21-hydroxylase gene lies on chromosome 6p21.3 among genes that code for proteins determining human leukocyte antigen (HLA) types. The 21-hydroxylase gene locus also has a pseudogene called CYP21A, which is about 30 kilobases (kb) away from CYP21A2. CYP21A is approximately 98% homologous in structure to CYP21A2, but is rendered inactive due to minor differences in the gene as compared to the active CYP21A gene. The proximity of CYP21A2 with CYP21A is thought to predispose the CYP21A2 to crossovers in meiosis between CYP21A2 and CYP21A or gene conversion, resulting in loss of gene function.

A number of point mutations and small deletions have been identified that deactivate this second copy. These differences include (in order from exons 1-10): P30L, IVS2-13 A/C>G (In2G), Δ8 bp, I172N, exon 6 cluster mutation (I235N, V236E, and M238K), V281L, F306+t, Q318X, and R356W. Transfer of any of these mutations from the pseudogene CYP21A to the functional CYP21A2 can occur through gene conversion or non-homologous recombination, thus inactivating the functional CYP21A2. Another source of mutation at the CYP21 locus is genomic rearrangement resulting in duplication or deletion of entire segments of the gene region. Large 30 kb deletions that fuse CYP21A with CYP21A2 account for approximately 25% of 21-hydroxylase deficiency. These types of mutation at the CYP21 gene locus account for approximately 90% of 21-hydroxylase deficiency. The remaining 10% of 21-hydroxylase deficiency is caused by rare sporadic mutation. The fact that this locus involves an evolutionary gene duplication produces technical difficulties for diagnosing mutation in the functional CYP21A2. Wedell & Luthman, Hum Genet (1993) 91:236-240; Speiser et al., Molecular Genetics and Metabolism, 71: 527-534 (2000); For a review see White & Speiser, Endocrine Reviews 21(3): 245-291 (2000).

The core problem in CAH is the inability of the adrenal glands to make enough cortisol in the non-salt wasting form, or enough cortisol and salt-retaining hormone in the salt-wasting form. Instead of making cortisol, the hormonal raw materials which usually make cortisol are shifted away to make other hormones, specifically male sex hormones (androgens). As a result more androgens are produced than necessary. Before birth, the excess androgens stimulate the growth of the genitalia. When the child is male this does not cause grave problems. However excess androgens in a female with this disorder causes the child's genitalia to have the appearance of a male although the internal genitalia are normal female. The assays of the present invention employ allele-specific amplification to determine a genotype of interest present in a sample of genomic DNA. While the present invention is described in terms of the CYP21 locus, the skilled artisan will understand that the methods described herein are applicable generally to gene loci in which two genes are subject to recombination resulting in variable genotypes present in a population. In various embodiments, the assays comprise four PCR reactions using four primer sequences in various combinations to detect genomic rearrangements at the CYP21 locus, including gene rearrangements and a large 30 kb gene deletion that results in a fusion gene.

In additional preferred embodiments, the assays also utilize primer extension methods in order to further characterize the 21-hydroxylase (CYP21) genotype present in the sample. While the methods are described in terms of the commercially available Applied Biosystems' SNaPshot™ method (Applied Biosystems, Foster City, Calif.), various primer extension methods are known to those of skill in the art. The exemplary methods described herein can detect both wildtype and mutant alleles at nine commonly mutated loci.

The basic strategy of the methods described herein involves amplifying genomic DNA. The resulting amplicon(s) are analyzed by agarose gel electrophoresis to determine which amplicons have been produced. After removing dNTPs and primers from the amplification products, the amplicon(s) are also subjected to a primer extension reaction by combining the PCR products, specific primers, and labeled nucleotides. An enzymatic reaction is then performed and unincorporated ddNTPs are removed. The samples are then analyzed by capillary electrophoresis and the data analyzed. Since each extension primer has a distinct molecular weight, and since each ddNTP has a distinct label (e.g., color) associated with it, the data is analyzed and particular labeled nucleotides are identified (e.g., by size and color) that correlate with the specific genotype present. Full sequencing of the CYP21A2 gene also is provided for genotyping as a backup to or in place of minisequencing primer extension.

By combining information regarding the size and number of amplicons produced, and the size of the extension primer as well as the fluorescent signal from the specific ddNTP that is added in the single base extension reaction, the specific genotype present in the sample is identified.

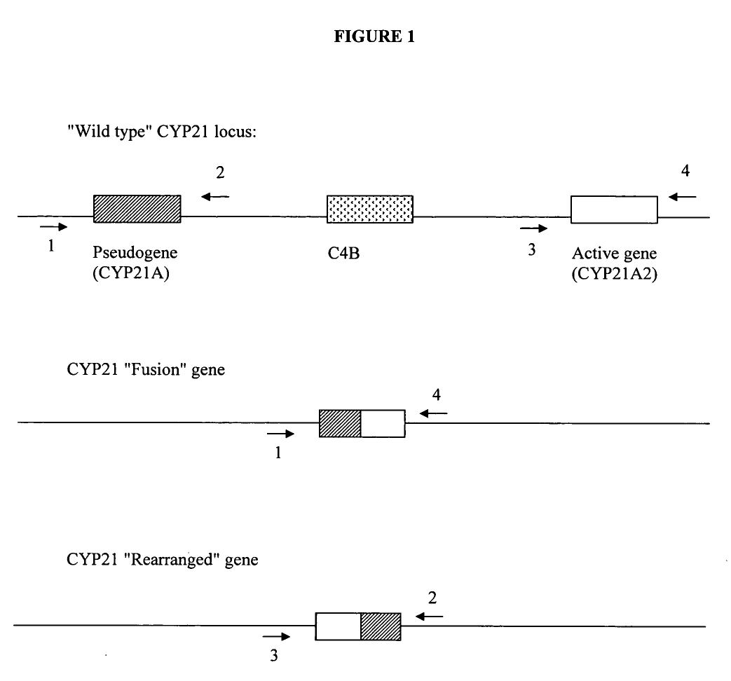

FIG. 1 provides a schematic illustration of the CYP21 gene region. Referring to FIG. 1 throughout, the active CYP21 gene is called CYP21A2 (or CYP21B in some nomenclature systems), while the duplicated gene (one copy evolutionarily inactivated) is called CYP21A (or the CYP21P pseudogene). If no deletion has occurred, primers 1 and 2 will amplify an approximately 4.0 kb fragment, and primers 3 and 4 will amplify an approximately 3.4 kb fragment. In this case primers 1 and 4 will be physically too far apart to yield a PCR product, and this amplicon will therefore not be detected by agarose gel electrophoresis. Homologous recombination between the active CYP21A2 and the pseudogene CYP21A may result in disease via deletion or gene conversion.

Unequal crossing over between the gene and pseudogene can cause deletion of a 30 kb fragment between the gene and pseudogene and enable production of amplicon with primers 1 and 4. When the deletion has occurred, the genomic sequence complementary to primers 2 and 3 have been removed from the chromosome. An approximately 4.0 kb product will be resolved by agarose electrophoresis.

When a gene rearrangement has occurred producing the reciprocal hybrid gene (A2/A), primers 3 and 2 can now form a PCR product. Thus a PCR product of approximately 3.4 kb will be resolved by agarose gel electrophoresis.

5′ gene conversion (movement of the primer 1 site ± other sequence from the pseudogene to the gene ) can also support amplification of the 4 kb product with primers 1 and 4. In this case, primer sites 2 and 3 would not be removed.

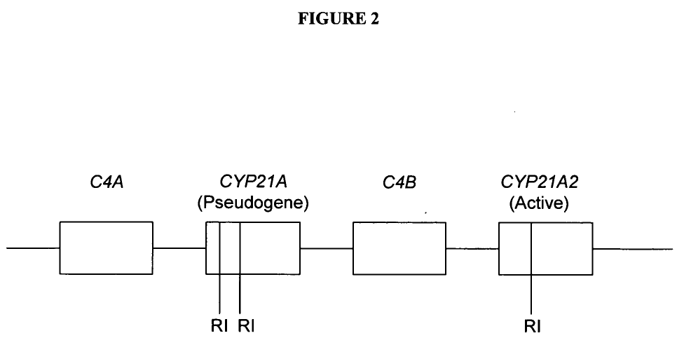

Confirmation of allele-specific amplification is provided by digestion with EcoRI. The CYP21A2 active gene contains one EcoRI restriction sites within its coding sequence while the CYP21A pseudogene contains two EcoRI restriction site. Therefore, referring to FIG. 2, digestion of the 3.4 kb amplicon containing the active CYP21A2 with EcoRI will produce two digestion products which can be distinguished by agarose gel electrophoresis as 1441-bp and 2579-bp fragments.

As shown in FIG. 2, digestion of the 4 kb amplicon containing the inactive CYP21A pseudogene with EcoRI will produce three products of 959-bp, 496-bp and 2579-bp. Digestion of the 4.0 kb A/A2 fusion amplicon or the 3.4 kb amplicon containing the A2/A rearrangement species may produce two or three digestion products, depending on where the breakpoint lies relative to the EcoRI sites.

The CYP21A pseudogene has P30L, IVS2-13A/C>G, Δ8 bp, I172N, exon 6 cluster mutation (I235N, V236E, and M238K), V281L, F306+t, Q318X, and R356W relative to the functional copy. The single base extension (SBE) of oligos that hybridize to the homologous (non-deletion) sequence in the active CYP21A2 are performed and analyzed, preferably with the SNaPshot™ minisequencing kit (Applied Biosystems). This analysis will confirm the presence or absence of the normal (non-mutation) CYP21A2 and/or mutation sequences.

Examples of apparatuses that may be useful for electrophoresis and visualization of amplicons are an agarose gel electrophoresis apparatus, such as CBS Scientific horizontal mini-gel; a power supply having a constant voltage of 100 to 200V or better variable power supply for electrophoresis, such as the BioRad Model 200; photodocumentation apparatus, such as the Alpha Innotech AlphaImager or Polaroid DS34 t; and a transilluminator, e.g., a VWR Model LM-20E or equivalent. Other methods of fractionating the amplification products can also be utilized, such as standard or HPLC chromatography methods, or nucleic acid hybridization microarrays.

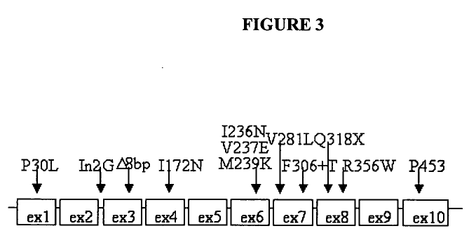

Approximately 70-75% of CYP21 disease cases are caused by eight common point mutations, small deletions, insertions, or conversion events. These genotypes can be determined by a number of different methods, all utilizing the 3.4 kb fragment obtained by allele-specific PCR utilizing primers 3 and 4 described above. Preferred primer extension primer locations and sequences are provided in Table 1 and FIG. 3, along with the genotype they are used to identify.

Numerous detectable labels for incorporation into nucleic acids are known to those of skill in the art. See, e.g., Handbook of Fluorescent Probes and Research Products, 9^{th} ed., Molecular Probes, Inc., 2002, Chapter 8 (“Nucleic Acid Detection and Genomics Technology”). Illustrative fluorescent labels include xanthene dyes, naphthylamine dyes, coumarins, cyanine dyes and metal chelate dyes, such as fluorescein, rhodamine, rosamine, the BODIPY dyes (FL, TMR, and TR), dansyl, lanthanide cryptates, erbium. terbium and ruthenium chelates, e.g. squarates, and the like. Additionally, in certain embodiments, one or more fluorescent moieties can be energy transfer dyes such as those described in Waggoner et al., U.S. Pat. No. 6,008,373. More recently, semiconductor-based quantum dots have also been described for use as biological labels (see, e.g., Bruchez et al., Science 281: 2013-16 (1998)); and mass spectrometry has been described for use in direct detection of unlabeled bases (see, e.g., U.S. Pat. No. 6,043,431)

The oligonucleotides generated by the primer extension methods described herein can be separated from each other so that each is mobilized in a manner that relates to each of their specific sizes. A preferred form of electrophoresis is capillary electrophoresis, but any form of electrophoresis that allows for the separation of a plurality of labeled nucleic acids in small volumes by automated or semi-automated methods and devices may be used. For high throughput of PCR products, an automated capillary electrophoresis (CE) system is used in order to separate labeled DNA molecules in a size-dependent manner, so that signals corresponding to each nucleotide in a sequence are detected in a sequential fashion. For reviews of the use of CE in DNA sequencing and polymorphism analysis, see Heller, Electrophoresis 22:629-43, 2001; Dovichi et al., Methods Mol. Biol. 167:225-39, 2001; Mitchelson, Methods Mol. Biol. 162:3-26, 2001; and Dolnik, J. Biochem. Biophys. Methods 41:103-19, 1999. In the Examples, the ABI PRISM® 3100 Genetic Analyzer may be used with an ABI PRISM 3100 capillary array, 36-cm (P/N#4315931). This provides a multi-color fluorescence-based DNA analysis system that uses capillary electrophoresis with 16 capillaries operating in parallel to separate labeled PCR products. A CE DNA sequencer/analyzer that operates 96 capillaries may be preferable in assays wherein 96-well plates are used. Analyzers with the capacity to process 96 wells include the MegaBACE™ 1000 DNA Analysis System (Molecular Dynamics, Inc and Amersham Pharmacia Biotech) and the 3700 DNA Analyzer from (Perkin-Elmer Biosystems)

The following examples serve to illustrate the present invention. These examples are in no way intended to limit the scope of the invention.

### EXAMPLE 1

### Preparation of Genomic DNA

Samples of whole blood were obtained from human subjects, and the blood samples processed to obtain samples of genomic DNA.

Many methods of isolating genomic DNA from blood are known. The following is one example: 10 ml of whole blood is added to 1 ml of proteinase K in a 50 ml tube. 10 ml of Guanidine HCl/Tween solution is added and the sample bump vortexed for 15 seconds. The sample is incubated at 65° C. for 10 min. and centrifuged briefly to remove any drops from the top of the lid. 10 ml of 100% ethanol is then added, the sample vortexed for 15 sec. and then centrifuged briefly to remove any drops from the top of the lid. 20 ml of the mixture is then transferred to the spin filter and centrifuged for 3 min at 2500×g. The remaining mixture is added to the spin filter and centrifuged for 3 min at 2500×g. The spin filter is transferred to a new 50 ml tube making sure ethanol is added to wash concentrate/Guanidine HCl solution before first use. 20 ml of wash concentrate/Guanidine HCl solution is added to the spin filter. The sample is centrifuged for 3 min at 2500×g. The spin filter is removed, the flow through discarded, and the spin filter replaced. 20 ml of wash concentrate/Tris, NaCl is added to the spin filter. The sample is centrifuged for 3 min 2500×g, the spin filter removed, the flow through discarded, and the spin filter replaced. The sample is centrifuged again for 3 min 2500×g. The spin filter is carefully removed and transferred to a new tube without coming in contact with the wash solution. 10 ml of 10 mM Tris-HCl is added. To increase yields, incubate for 5 min. at 65° C. The tube is centrifuged for 3 min. at 2500×g. The filter unit is removed and the tube lid closed. Genomic DNA in the tube is now ready to use for any application.

In another embodiment the genomic DNA can be isolated by other procedures. For example the KingFisher™ Blood DNA Kit by Thermo LabSystems™ (Vantaa, Finland) provides another commercially available method for isolating genomic DNA. The protocol ensures that only those blood cells containing DNA are isolated for purification. The first step utilizes Pan Leucocyte Dynabeads™ (Dynal Biotech, Oslo, Norway) to isolate the white blood cells. Dynabeads M-450 CD2 are uniform, magnetizable polystyrene beads coated with a primary monoclonal antibody (mAb) specific for the CD2 membrane antigen which is predominantly expressed on human T cells. The mouse IgG1 mAb is attached to Dynabeads™ via a secondary antibody to effect optimal orientation of the primary antibody. Dynabeads™ CD2 are supplied as a suspension containing 4×10⁸ beads/ml in phosphate buffered saline (PBS), pH 7.4, containing 0.1% bovine serum albumin (BSA) and 0.02% sodium azide (NaN₃). In this way, all impurities and unwanted material, such as red blood cells, are left behind. The purified white blood cells are then washed, lysed and mixed with DNA-binding Dynabeads™ which bind the genomic DNA. Subsequent washing steps remove unwanted cell debris and other contaminants, resulting in an average yield of 4-10 μg of highly purified DNA from 200 μl of blood. DNA can be eluted either into microcentrifuge tubes or another suitable vessel.

In some examples, DNA was extracted from patient peripheral blood samples or cultured amniocytes using the QIAamp DNA kit (Qiagen, Valencia, Calif.).

### EXAMPLE 2

### Preparation of Master Mixes For CAH Analysis

Each master mix provides the primers necessary for carrying out the four PCRs that will determine the result of the assay. The master mixes that are used in the PCR procedure were prepared as follows:

Master Mix #1 (CYP21A2 Amplification)

| Components | for 1 Rxn | | for 110 Rxns | |
|---|---|---|---|---|
| H₂O | 29.6 | μL | 3256 | μL |
| 10× Qiagen ™ PCR buffer | 5.5 | μL | 605 | μL |
| 5× Q solution | 10.0 | μL | 1100 | μL |
| 25 mM dNTP mix | 0.5 | μL | 55 | μL |
| 10 μM primer 3 | 1.5 | μL | 165 | μL |
| 10 μM primer 4 | 1.5 | μL | 165 | μL |
| 5 μM AVPR2 primer mix* | 1.5 | μL | 165 | μL |
| Total | 50.0 | μL | 5511 | μL |

Master Mix #2 (CYP21A Amplification)

| Components | for 1 Rxn | | for 110 Rxns | |
|---|---|---|---|---|
| H₂O | 29.6 | μL | 3256 | μL |
| 10× Qiagen ™ PCR buffer | 5.5 | μL | 605 | μL |
| 5× Q solution | 10.0 | μL | 1100 | μL |
| 25 mM dNTP mix | 0.5 | μL | 55 | μL |
| 10 μM primer 1 | 1.5 | μL | 165 | μL |
| 10 μM primer 2 | 1.5 | μL | 165 | μL |
| 5 μM AVPR2 primer mix* | 1.5 | μL | 165 | μL |
| Total | 50.0 | μL | 5511 | μL |

Master Mix #3 (30 kb Deletion Product Amplification)

| Components | for 1 Rxn | | for 110 Rxns | |
|---|---|---|---|---|
| H₂O | 29.6 | μL | 3256 | μL |
| 10× Qiagen ™ PCR buffer | 5.5 | μL | 605 | μL |
| 5× Q solution | 10.0 | μL | 1100 | μL |
| 25 mM dNTP mix | 0.5 | μL | 55 | μL |
| 10 μM primer 1 | 1.5 | μL | 165 | μL |
| 10 μM primer 4 | 1.5 | μL | 165 | μL |
| 5 μM AVPR2 primer mix* | 1.5 | μL | 165 | μL |
| Total | 50.0 | μL | 5511 | μL |

Master Mix #4 (Genomic Rearrangement Product Amplification)

| Components | for 1 Rxn | | for 110 Rxns | |
|---|---|---|---|---|
| H₂O | 29.6 | μL | 3256 | μL |
| 10× Qiagen ™ PCR buffer | 5.5 | μL | 605 | μL |
| 5× Q solution | 10.0 | μL | 1100 | μL |
| 25 mM dNTP mix | 0.5 | μL | 55 | μL |
| 10 μM primer 3 | 1.5 | μL | 165 | μL |
| 10 μM primer 2 | 1.5 | μL | 165 | μL |
| 5 μM AVPR2 primer mix* | 1.5 | μL | 165 | μL |
| Total | 50.0 | μL | 5511 | μL |

*AVPR2 primers are AVPR2F: SEQ ID NO 33: 5′-TGACCATCCCTCTCAATCTTC-3′; and AVPR2R: SEQ ID NO 34: 5′-TCCCTCTTTCCTGCCACTCCT-3′.

A sufficient quantity of Taq polymerase was added to master mix tubes. While any suitable Taq polymerase can be used, in a preferred embodiment HotStarTaq™ is utilized (Qiagen, Valencia, Calif.). HotStarTaq™ is a modified form of Taq DNA Polymerase. It is supplied in an inactive state that has no polymerase activity at ambient temperatures. This prevents extension of nonspecifically annealed primers and primer-dimers formed at low temperatures during PCR setup and the initial PCR cycle. HotStarTaq™ DNA Polymerase is activated by a 15-minute incubation at 95° C. which can be incorporated into any existing thermal-cycler program.

HotStarTaq™ Master Mix is a premixed solution containing HotStarTaq™ DNA Polymerase, PCR Buffer, and dNTPs. The solution provides a final concentration of 1.5 mM MgCl₂ and 200 μM of each dNTP. The PCR buffer contains a combination of KCl and (NH₄)₂SO₄ in the buffer to promote specific primer-template annealing and reduce nonspecific annealing, thereby maximizing yields of specific PCR product.

A PCR additive is also used in the most preferred embodiments to assist in extending PCR. The preferred PCR additive is Taq Extender™ PCR additive, which improves the reliability and yield of conventional Taq-based PCR amplifications. Taq Extender™ additive increases the efficiency of Taq DNA polymerase extension reactions during each cycle of PCR by reducing mismatch pausing, thus resulting in a greater percentage of completed extension reactions. In addition, Taq Extender™ PCR additive improves the PCR amplification of difficult templates and increases the reliability and yield of many PCR targets up to 35 kb in length. The additive is simply added to amplification reactions in an amount equal to that of Taq DNA polymerase, the standard buffer is replaced with an optimized 10×Taq Extender™ buffer and standard PCR cycling conditions are used. Taq Extender™ buffer contains 200 mM Tris-HCl (pH 8.8), 100 mM KCl, 100 mM (NH₄)2SO₄, 20 mM MgSO₄, 1% Triton X-100, and 1 mg/ml nuclease-free bovine serum albumin. Nielson, K. B., et al. (1994) Strategies 7: 27; Innis et al., (1988) Proc Natl Acad Sci USA, 85(24): 9436-40.

The person of ordinary skill will realize that many other Taq polymerases are commercially available or that can be manufactured. For example, another embodiment utilizes SureStart™ Taq DNA polymerase, which is commercially available (Stratagene, La Jolla, Calif.). The enzyme can also be used effectively in amplification reactions to achieve reduced background and high yield of PCR product. The enzyme consists of a reversibly inactivated DNA polymerase that remains inactive until stringent denaturation temperatures are reached. It is activated by adding a heat step (9 to 12 minutes at 92 to 95° C.) to the beginning of thermal cycling programs (pre-PCR heat-activation method). Alternatively, the pre-PCR heat-activation step can be omitted to permit enzyme activation during temperature cycling. When this method is employed, it is preferable to add additional cycles to existing cycling programs to achieve optimal product yield.

The above master mixes were also run with an AVPR2 internal control primer set, 5′-ACAGGCTCTGGCCAATTCTC-3′ (SEQ ID NO: 35); 5′-ACCTGGCCGTGGCTCTGTTC-3′ (SEQ ID NO:36). The product of these primers is 1.1 kb.

### EXAMPLE 3

### PCR Amplification for CAH Analysis

A template is created showing the position of each sample/control to be amplified and identifying which samples receive which Master Mix and other ingredients.

For each sample four PCR reactions were set up. A Master Mix, HotStarTaq™, Taq Extender™, and DNA were added to the wells of a 96 well plate. The plate was vortexed (without creating bubbles) and transferred to a thermal cycler (Perkin-Elmer GeneAmp 9700, Perkin-Elmer Instruments, Boston, Mass.). The reaction was then started. The program for the thermal cycler was as follows: 95° C. for 5 minutes followed by: 10 cycles of 95° C. for 30 seconds, 65° C. for 30 seconds (temperature reduced by 1° C. each cycle), 72° C. for 2 minutes; and 35 cycles of 95° C. for 30 seconds, 55° C. for 30 seconds, 72° C. for 2 minutes, with a final extension at 72° C. for 10 minutes. Reactions can beheld overnight at 10° C.

### EXAMPLE 4

### Agarose Electrophoresis for CAH Analysis

The PCR amplicons were assayed on a 0.7% agarose gel. Amplification was confirmed by the presence of a 2 kb internal control band (or 1.1 kb). In some experiments, electrophoresis was performed with a 1% agarose gel. Approximate sizes of amplicons as compared to the size of the standard are:
CYP21A2=3.4 kb
CYP21A=4.0 kb
Hybrid 1=4.0 kb (30 kb deletion product of homologous recombination or gene conversion without deletion—A/A2)
Hybrid 2=3.4 kb (reciprocal rearranged gene—A2/A)
Internal Control=2.0 kb (or 1.1 kb for SEQ ID NOs: 35 and 36)

5 μl of TE buffer (0.01 M Tris, 0.05 M EDTA, pH 7.5) was added to 5 μl of PCR product. For the standard lane, 1 μl of 1 kb ladder and 10 μl of TE was combined. 2 μl of 6× loading buffer (Blue/Orange 6× loading dye (Promega, Madison, Wis.)) was added to all wells and the sample electrophoresed at 100 V for about 2 hours.

### EXAMPLE 5

### Single Base Extension for CAH Analysis

Following PCR, Shrimp Alkaline Phosphatase (SAP) and Exonuclease I (Exo I) are preferably used to prepare PCR products for base extension. SAP removes the phosphate groups from the excess dNTPs left over from the PCR reaction, and Exonuclease I digests the single stranded PCR primers into dNTPs. Both enzymes are readily inactivated by heating at 75° C. for 15 min and therefore do not affect the single base extension reaction.

Two μl of amplicon was digested with two units of SAP and one unit of ExoI where were added to 1× SAP buffer to a final volume of 6 μl per reaction. The parameters for the Exo/SAP reaction were 37° C. for 2 hours, 75° C. for 15 minutes, and hold at 4° C. SAP buffer contains 25 mM Tris-HCl, 10 mM MgCl₂, (pH 9.0 at 37° C.).

The primers included extension primers listed in Table 1 and were prepared according to Table 2:

TABLE 3

| | Primer Stock | Primer Final | | | |
|---|---|---|---|---|---|
| Primer | Concentra- | Concentra- | Peak | Wildtype | Mutant |
| Location | tion (μM) | tion (μM) | size | peak | peak |
| P30L | 100 | 3 | 22-24 | blue | green |
| I2GIVS2 - | 100 | 8 | 29-30 | red/blue | black |
| I172N | 100 | 8 | 45-46 | green | red |
| V281L | 100 | 1 | 68-70 | blue | red |
| Q318X | 100 | 8 | 80-84 | black | red |
| R356W | 100 | 10 | 88-90 | black | red |
| I235N | 100 | 11 | 50-52 | red | green |
| V236E | 100 | 13 | 57-59 | green | red |
| M238K | 100 | 12 | 61-63 | green | red |
| F306+t | 100 | 0.5 | 76-78 | blue | red |
| Δ8bp-F | 100 | 8 | 34-38 | blue | red |
| Δ8bp-R | 100 | 6 | 39-41 | blue | black |
| P453S | 100 | 10 | 93-95 | black | red |

5 μl of reaction mix containing the distinctively labeled ddNTPs and 1 unit of DNA polymerase in a 20 μl reaction volume of buffer (10 mM KCl, 20 mM Tris-HCl (pH 8.8 at 25 C), 10 mM (NH₄)₂SO₄, 2 mM MgSO₄ and 0.1% Triton X-100), 5 μl of SAP/ExoI-treated PCR product, and 1 μl of primer extension mix was added to a fresh 96 well tray (or in strip cap 0.2 μl tubes). The plate was sealed and vortexed to mix, with a quick spin to collect samples. The plate was transferred to the thermal cycler and subjected to the following parameters for single base extension: Denature 96° C. for 30 seconds, Anneal 50° C. for 5 seconds, Elongate at 60° C. for 30 seconds, repeat for 25 cycles, hold at 10° C. overnight.

A second SAP reaction was then performed by adding 1 μl of SAP to each well. The plate was sealed, vortexed to mix, and quick spun to collect samples. The plate was then transferred back to the thermal cycler and subjected to the following parameters: 37° C. for 1 hour, 75° C. for 15 minutes, and hold at 4° C.

### EXAMPLE 6

### Capillary Electrophoresis and Detection for CAH Analysis

Capillary electrophoresis is an analytical technique that separates species by applying voltage across buffer filled capillaries. It is generally used for separating ions, which move at different speeds when the voltage is applied depending on their size and charge. The solutes are seen as peaks as they pass through the detector and the area of each peak is proportional to their concentration, which allows quantitative determinations. Analysis times are in the region of 1-30 minutes depending on the complexity of the separation. Modern instruments are relatively sophisticated and may contain fibre optical detection systems, high capacity autosamplers, and temperature control devices. Detection is usually by UV absorbance, often with a diode array. Fluorescence detection is the preferred mode of detection. Indirect UV detection is widely used for detecting solutes having no chromophores such as metal ions or inorganic anions. Low UV wavelengths (e.g., 190-200 nm) are also used to detect simple compounds such as organic acids.

An ABI PRISMS 3100 Genetic Analyzer (Applied Biosystems, Foster City, Calif.) was used for the capillary electrophoresis analysis. The ABI PRISM® system is a fluorescence-based, fully automated DNA analysis system using capillary electrophoresis with 16 capillaries operating in parallel. Samples were prepared by mixing 1 μl of each sample with 20 μl of Hi-Di™ formamide (Applied Biosystems, Foster City, Calif.) and 1 μl of LIZ™ internal size standard (Applied Biosystems) in a 96-well tray or in strip cap 0.2 μl tubes. The wells were covered and the plate vortexed to mix. The plate was then heated at 92-95 ° C. for 3-5 minutes in a heat block, and then transferred to ice. The ABI PRISM® was set up and operated according to the manufacturer's instructions.

The SNaPshot™ Multiplex System consists of the SNaPshot™ Multiplex Kit, together with GeneScan™ 120 LIZ™Size Standard, Genotyper® Software version 3.7, together with the ABI PRISM® capillary electrophoresis instrument (all from Applied Biosystems). This system is commercially available and is a preferred embodiment for high-throughput SNP validation and medium-throughput linkage and association studies. Thousands of SNPs per day can be determined using this system.

The assay system allows multiplexing during single base extension (SBE) of at least 13 primer-template combinations in a single tube/single capillary format. Therefore it is highly preferred for the determination of which nucleotide is added in the present application. Separation of SNP loci can be determined down to one base-pair. In the single base extension reaction primers that bind to particular nucleic acid sequences are extended by a single nucleotide. The ddNTPs are provided in the reaction, and each type of ddNTP is provided with a distinct fluorescent label in the preferred embodiment.

Therefore, it is determined that the combination of the molecular weight differences in the extension primers (due to the poly-T tail) and the fluorescent labeling of the single base extension reactions enables the precise identification of the genotype at. the CYP21 gene. Specific primers have been correlated with specific genotypes. Because those primers have been engineered as described herein to have different molecular weights (conferred by the differing poly-T tails attached to them), one level of identification is obtained by identification of the presence of a labeled primer. Through the single base extension reaction the identity of particular nucleotides is also determined, thereby enabling the user to definitively identify the primers present and specifically match those primers to particular genotypes. Determination of the genotypes is preferably done in an automated fashion using a suitable software, such as the Genotyper® software.

After CE and analysis with GeneScan™ and GenoTyper™ software (Applied Biosystems), non-overlapping peaks with similar heights are produced that provide genotype information. Poly-T chains at the 5′ end of most oligonucleotides (Table 1) prevent the resulting peaks from overlapping, and help reduce secondary structure formation due to the addition of other sequences. The small 8-bp deletion commonly found in exon 3 was analyzed by two SBE reactions that add bases in the forward and reverse directions (Δ8 bp-F and Δ8 bp-R). To facilitate rapid scoring, the genotyper software was programmed to label peaks representing wild-type alleles as black, and peaks representing mutant alleles as red. A minisequencing method and reporting chart also provides easy-to-read break-point information for gene deletions and large conversion events when a series of black labels change to a series of red labels, or vice versa.

While the techniques describe above are the most preferred embodiment, in other embodiments other techniques can be used to determine the genotypes. In one embodiment the Taqman™ Allelic Discrimination system (Applied Biosystems, Foster City, Calif.) can be used instead of the SNaPshot™ system. TaqMan® Pre-Developed Assay Reagents for Allelic Discrimination (PDARs for AD) are available for the ABI PRISM® 7700 (Applied Biosystems) and the ABI PRISM® 7900 HT Sequence Detection Systems (Applied Biosystems). The TaqMan® PDARs for AD use the 5′ nuclease assay to genotype purified DNA samples for specific mutations. Most TaqMan® PDARs for AD assays discriminate between two alleles of single nucleotide polymorphisms (SNPs). Each assay contains two different TaqMan® probes, and each uniquely labeled probe binds preferentially to one of the alleles.

The TaqMan® probes contain two additional features that provide better allelic discrimination. The minor groove binder (MGB) enhances the discrimination between match and mismatch by allowing the use of shorter probes. Second, a non-fluorescent quencher allows better spectral discrimination between the reporter dyes.

TaqMan® PDARs for AD are convenient to use because reaction set-up involves only four components including the sample. Since genotyping is done by endpoint reading the TaqMan® PDAR reaction, high-volume users can use multiple thermal cyclers to amplify DNA before the cycled reactions are analyzed.

In still more embodiments, other methods of genotyping the mutants can be used. As an alternative to the methods described above, primer extension with fluorescently labeled ddNTPs can also be performed on widely available automated sequencers. One variation of the methods above uses extension primers labeled with 3 different dyes (FAM, HEX, and TET). Size standards can be designed ranging from 13 to 43 bp, labeled with TAMRA. In one embodiment, the 3′ end of the primer extends not immediately up to the SNP, but one bp earlier. This reduces the interference of the first extension peak with the peak produced by the primer which has not incorporated any d/ddNTP. Three dNTPs and 1 ddNTP can be used for each reaction, so that the primer extends between 1 and 5 bp beyond the SNP, until it reaches the next ddNTP site. This produces 3 peaks which allow unambiguous genotype determination. The presence of a peak produced by the non-extended primer in each reaction allows failed reactions to be distinguished and provides an internal size control. Using different sizes of primers, ranging from 15 to 31 bp, 8 SNPs can be multiplexed for analysis on the same gel at low cost.

### EXAMPLE 7

### Cycle Sequencing CYP21A2

CYP21A2 mutations not detectable using primer extension, including L433P, R483+C, R408C and W407X/null, can be identified by sequencing the complete CYP21A2 encoding gene using cycle-sequencing in forward and reverse directions with primers SEQ ID NOs: 18-32 (table 2). PCR products were Exo/SAP-purified (as described above) were cycle-sequenced using the primer set SEQ ID NOs: 18-32 and Big Dye, Version 3.1 (Applied Biosystems), according to the manufacturer's protocol. Sequences were aligned and examined using SeqScape software (Applied Biosystems).

### EXAMPLE 8

### Results and Analysis

Typical samples from normal individuals produce amplicons 1 (CYP21A2) and 2 (CYP21A), but may also produce amplicon 4 (CYP21A2/A). This atypical result has been observed on multiple occasions, and may correspond to a cis gene duplication. Alternatively, amplicon 4 with normal CYP21A2 alleles may be the result of gene conversion at the 3′ end of the gene, which does not cause a coding change. Minisequencing results for wild-type amplicon 1 yield non-overlapping peaks of similar sizes with all-black labels. Minisequencing results for the pseudogene in amplicon 2 may be non-overlapping mutant peaks, with all-red labels. However, much heterozygosity has been observed within the pseudogene amplicon, so the peaks and labels may reflect a mixture of wild type and mutant. Minisequencing results for wild-type amplicon 4, if present, produce an identical pattern to that of amplicon 1.

Typical samples from individuals affected by CAH or carriers may produce any combination of the four PCR amplicons. The presence of amplicon 3 indicates a 30-kb deletion allele or a large gene-conversion allele with the 5′ end of CYP21A and the 3′ end of CYP21A2. The presence of amplicon 4 may indicate a normal allele, a gene-conversion event or another mutant, all of which can be distinguished by the presence or absence of mutant alleles within the minisequencing results. Minisequencing results for amplicon 1 exhibit non-overlapping peaks, with one or more mutant red-labeled peaks. As in samples from normal individuals, amplicon 2 SNaPshot™ results indicate much heterogeneity. Gene conversion or large deletion events and their breakpoint locations within amplicons 3 or 4 are indicated by a trend of red-labeled peaks followed by a trend of black-labeled peaks, or vice versa. Care should be taken when considering the peak for P453S, because this mutation is not transferred from the pseudogene and may be mistakenly identified as the tail end of a gene-conversion event. The presence of a mutation at one or more loci within amplicon 4 is determined by the presence of one or more mutant red-labeled peaks, similar to amplicon 1.

Twenty samples from CAH patients or carriers and 18 samples from healthy donors previously characterized for CYP21 mutations were analyzed during assay validation (Table 4). Clinical subtypes of the patients (if available) were determined based on clinical manifestations and the levels of relevant steroid metabolites and electrolytes in plasma and urine. See Wilson et al., J Clin Endocrinol Metab 80:2322-2329 (1995). Mutations from 15 of the CAH patient or carrier samples were previously determined using Southern hybridization for deletion analysis, allele-specific PCR for detection of the eight most common point mutations (see J Clin Endocrinol Metab 1995, 80:1635-1640), and sequencing for the detection of remaining mutations. Three samples with a V281L mutation were collected at Quest Diagnostics for validation of the CAH Ashkenazi Jewish allele assay, and to carry V281L mutations using a READIT™ assay (Promega Corporation, Madison, Wis.). Mutations from the two remaining samples from CAH patients were previously determined by allele-specific PCR only. The validation panel was assessed for the presence of 13 mutations (the 30-kb deletion, 11 point mutations and one small deletion), and had 11 different mutant CYP21A2 genes. Each of the 13 mutations examined in the assay was detected at least once.

TABLE 4
Assay Validation Panel*

| Sample | Expected genotype^{†} | Observed genotype^{‡} | Phenotype |
|---|---|---|---|
| 4110 | In2G/F306+T | In2G/F306+T | Unknown |
| 5846 | 30-kb del/gene | 30-kb del/gene | Salt wasting CAH |
| | conversion | conversion | |
| 5686 | Null/null | Null/null | Salt wasting CAH |
| 5557 | In2G/Q318X | In2G/Q318X | Severely virilized |
| | | | CAH |
| 6025 | In2G/R356W | In2G/R356W | Salt wasting CAH |
| 7214 | In2G/I172N | In2G/I172N | Severely virilized |
| | | | CAH |
| 7215 | In2G(A, C)/Q318X | In2G(A, C)/Q318X | Unaffected carrier |
| 7227 | In2G/null^{§} | In2G/In2G^{§} | Salt wasting CAH |
| 7229 | WT/In2G | WT/In2G | Unaffected carrier |
| 7243 | V281L/30-kb del | V281L/30-kb del | Non-classic CAH |
| 7257 | V281L/V281L | V281L/V281L | Non-classic CAH |
| 3904 | In2G/L433P | In2G/WT [symbol] | Unknown |
| 4451 | In2G/P453S/R483+C | In2G/P453S [symbol] | Unknown |
| 5467 | Δ8bp/R408C | ASbp/WT [symbol] | Unknown |
| 6730 | W407X/null | WT/WT [symbol] | Unknown |
| RD03-1 | V281L/V281L | V281L/V281L | Non-classic CAH |
| RD03-3 | V281L/V281L | V281L/V281L | Non-classic CAH |
| RD03-5 | WT/V281L | WT/V281L | Unaffected carrier |
| 147 | WT/V281L/gene | WT/V281L/gene | Unaffected carrier |
| | conversion | conversion | |
| 46500 | WT/V281L/gene | WT/V281L/gene | Non-classic CAH |
| | conversion | conversion | |

*The PCR/minisequencing assay correctly typed 18 samples as wild type (data not shown). Wild-type samples included one anonymized prenatal sample.
^{†}Determined at Cornell University using Southern hybridization/allele-specific PCR, reflexed to a sequencing assay when indicated, or at Quest Diagnostics using a READIT ™ assay (Promega Corporation).
^{‡}Determined by PCR/minisequencing assay.
^{§}Both the expected and observed genotypes were interpreted as being consistent with a CAH phenotype. [symbol]
The expected genotype includes one or more mutations not detected in the PCR/minisequencing assay. These mutations were subsequently detected in an assay that sequences the entire CYP21A2 gene.

Using the methods disclosed herein, all validation samples were correctly typed, according to the expected genotypes. Results from seven samples warrant further discussion. Amplicon 1 of sample 7215 had A, C and G alleles at the In2G locus, revealing the presence of at least three copies of the CYP21A2 gene. Although the individual carries both In2G and Q318X mutations, it was suspected of being unaffected because of the third copy of CYP21A2. The laboratory that contributed the sample confirmed the presence of the third normal allele.

Samples 3904, 4451, 5467 and 6730 all contain mutant alleles that are not detected by primer extension minisequencing. These mutations were subsequently detected by complete gene sequencing as described above.

Samples 147 and 46500 are from an asymptomatic mother and her son with NC disease characterized by precocious pubarche and advanced bone age. Minisequencing as well as complete gene sequencing failed to find a CYP21A2 mutation.

Over 100 clinical samples had been analyzed for CYP21A2 mutations using the methods disclosed herein. The majority of samples (n=95; 93%) were from peripheral blood, but seven (7%) were prenatal samples from cultured amniocytes. Prenatal samples were tested only after confirmation of the indication by a genetic counselor, and were tested in duplicate to confirm the analytical results.

The majority of patients (n=53) submitted for testing were suspected of having CAH (either classic or NC disease), based on physical or biochemical examination. This group included samples from seven prenatal subjects, whose parents are obligate carriers (1 in 4 risk of disease). The remaining samples were from virilized infants or children (n=12), those submitted for confirmation of a previous diagnosis (n=9) or those suspected of having NC disease (n=25); 19 (36%) of these suspected samples had no normal CYP21A2 alleles. A further eight patients (15%) were carriers (one normal allele with one or more mutant alleles). A total of 26 patients (49%) carried no mutations, according to the current method, and may be reflexed to a sequencing protocol if clinically indicated.

In total, 49 samples were submitted from individuals with a family history of CAH, including parents of affected children (obligate carriers), siblings of affected individuals and individuals with other family histories, including spouses, cousins, nephews, etc. A total of 18 samples from parents of a previous affected child (obligate carriers) were submitted for testing; two of these carried no normal CYP21A2 alleles, although they did not previously know they were affected. An additional 13 samples were found to be carriers, and three were wild type with at least one normal allele. Three samples from siblings of affected individuals were tested; two of these were found to be carriers, and the third had at least one normal allele. Eighteen samples were submitted from individuals with other family histories; this group comprised two carriers and 16 normal individuals.

The final group of samples (n=10) were submitted for testing without giving an indication for testing, or for R&D purposes. No patient in this group was affected (i.e. no abnormal CYP21A2 alleles), but two (18%) carried mutations.

The inclusion of 11 point mutations and one small deletion in the minisequencing assay, as well as the testing of several families and triads for prenatal diagnostics, allowed observation of several unusual haplotypes that maybe useful for clinical diagnosis. During validation, seven individuals with a V281L mutation were tested. Two of these, 7257 and 7243, were samples from Ashkenazi NC CAH patients originally diagnosed by Southern blot analysis and locus-specific PCR. In the assay, both samples produced amplicons 2, 3 and 4, with identical minisequencing results for amplicons 2 and 4, but different results for amplicon 3. Amplicon 3 of sample 7257 is mutant for P30L, In2G, Δ8 bp, I172N, I235N, V236E, M238K, V281L and F306+t, and wild type for Q318 and R356, indicating a deletion/conversion fragment with an intron 7 breakpoint. In contrast, minisequencing results from the product of PCR reaction 3 for sample 7243 revealed heterozygosity and, therefore, the presence of two or more amplicon 3 fragments. These amplicons were homozygous mutant for P30L, In2G and Δ8 bp, heterozygous for I172N, I235N, V236E, M238K, V281L and F306+t, and homozygous wild type for Q318 and R356, indicating that one has an intron 3 breakpoint and the second has an intron 7 breakpoint. Southern blot results indicate the presence of a duplicated gene fragment in both samples (intron 7 breakpoint), while sample 7243 additionally carries a 30-kb deletion allele with an intron 3 breakpoint. The amplicon 3 fragment probably corresponds to a gene duplication with a large conversion event. Whether the product of deletion or conversion, the presence of multiple mutant alleles within this fragment indicates that it will not produce functional protein.

Another unusual haplotype that was observed involves a single chromosome with two copies of CYP21A2: one wild-type copy and a second carrying the Q318X mutation. This chromosome was first observed in sample 7215 during validation (discussed above), and was observed in a further four individuals from two families. Although the assay facilitates observation of the second copy of CYP21A2 through A/C heterozygosity at the IVS2-13 locus, the Q318X mutation could be misinterpreted in an assay that does not identify two separate chromosomes.

The 21-hydroxylase deficiency molecular diagnostic assay described herein provides a reliable and robust method to detect common mutations within CYP21A2, including large genomic rearrangements, point mutations and a small 8-bp deletion. This system allows rapid and accurate locus-specific identification of deletions and conversions. The assay functions reliably in routine clinical testing and is sufficiently efficient to allow prenatal testing. The assay allowed a rapid turnaround from receipt of the clinical blood sample or cultured prenatal sample to written report of the diagnostic result.

The relatively compact size of the two genes (3.1 kb) allowed amplification of each gene accurately, by taking advantage of the limited heterogeneity outside of the coding region. The cis arrangement of CYP21A2 and CYP21 on a single chromosome allowed detection of large deletions or conversions without a Southern blot, by combining the primers into all four possible pair-wise combinations. Furthermore, inclusion of an internal control amplicon decreases the likelihood of false-positives, by controlling for PCR reaction conditions.

The minisequencing used herein is multiplexed, semi-automated and efficient, providing simultaneous detection of 11 point mutations and a small 8-bp deletion within a single tube. CE detection of labeled oligonucleotides allows accurate sizing/discrimination, and color-coded labels aid mutation and breakpoint reporting. This approach is clinically appealing because it reduces the total number of PCR reactions, compared with other methods, and uses fluorescent labeling of oligonucleotides and CE instead of radioactive labeling, as is optimally required in Southern blots.

The results to date demonstrate sensitivity and specificity of the assay. Although one would expect to find mutations on both chromosomes in 100% of known affected individuals, only 19/53 patients (36%) submitted for suspected disease had two mutations. This finding is likely to be due to physicians submitting samples simultaneously for both biochemical and molecular testing. In an academic setting, patients might be subjected to a more linear testing pattern; however, the commercial laboratory allows physicians the freedom to test as clinically indicated. Results herein for prenatal samples are limited, but are closer to the expected pattern of 1/4 affected, 1/2 carrier and 1/4 unaffected—found were 2/7 (28%) affected, 4/7 (57%) carriers and 1/7 (14%) unaffected. These findings for obligate carriers are also close to the expected carrier rate of 100%, with 15/18 (83%) being carriers or affected. 2/3 normal siblings of affected children were found to carry a mutation. Individuals without a family history (including spouses of affected individuals or carriers without other previous family history) would be expected to have only a 1/60 chance of carrying a classic mutation or a 1/15 chance of an NC mutation.

Examination of Ashkenazi V281L mutant chromosomes reveals the possibility that amplicon 3 may be interpreted as a 30-kb deletion when it is actually a gene duplication with a large conversion event. Therefore, it may be difficult in practice to identify deletions without analysis of an entire family. Southern blots using both the CYP21 and C4 probes may provide added information about the presence or absence of a deletion. Regardless of whether the rearrangement event is the product of conversion or deletion, the minisequencing component of this method allows determination that amplicon 3 associated with the V281L allele is non-functional, by examining the pattern of mutations.

The assay described herein has been optimized for use as a qualitative diagnostic tool. In instances where the IVS2-13 A/C>G locus is not multiply heterozygous, or two CYP21A2 genes do not amplify independently into amplification reactions 1 and 4, the presence of a functional gene may be difficult to detect. Therefore, difficult cases involving compound heterozygosity may require additional family studies. Prenatal diagnosis may also warrant further family studies, to establish the phase of multiple mutations.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A method of determining a CYP21 genotype in a sample comprising DNA, comprising,
(a) amplifying the DNA using four amplification primers to produce
(i) a first amplicon corresponding to a full length CYP21A gene if present, utilizing a first primer and a second primer selected to produce the first amplicon in the presence of the full length CYP21A gene, but not in absence of the full length CYP21A gene,
(ii) a second amplicon corresponding to a full length CYP21A2 gene if present, utilizing a third primer and a fourth primer selected to produce the second amplicon in the presence of the full length CYP21A2 gene, but not in the absence of the full length CYP21A2 gene,
(iii) a third amplicon corresponding to a CYP21A/CYP21A2 fusion gene if present utilizing one of said first primer and second primer and one of said third primer and fourth primer to produce the third amplicon in the presence of the CYP21A/CYP21A2 fusion gene, but not in absence of the CYP21A/CYP21A2 fusion gene, and
(iv) a fourth amplicon corresponding to a CYP21A2/CYP21A rearranged gene if present utilizing one of said first primer and second primer and one of said third primer and fourth primer to produce the fourth amplicon in the presence of the CYP21A2/CYP21A rearranged gene, but not in absence of the CYP21A2/CYP21A rearranged gene;
(b) determining which of the first, second, third, and fourth amplicons are produced;
(c) determining the genotype in the amplicons by using two or more oligonucleotides selected from SEQ ID NOs 5-17 or from SEQ ID Nos 18-32.

2. The method of claim 1 wherein step (c) use of SEQ ID NOs 5-17 is achieved by primer extension with distinctively labeled ddNTP from a set of ddNTPs.

3. The method of claim 1 wherein the ddNTPs comprise a fluorescent label.

4. The method of claim 2 wherein the set of ddNTPs comprise ddATP, ddCTP, ddGTP, and ddTTP, each of which is labeled with a distinct fluorescent label.

5. The method of claim 1 wherein
the first amplicon is a PCR product of about 4.0 kb in length;
the second amplicon is a PCR product of about 3.4 kb in length;
the third amplicon is a PCR product of about 4.0 kb in length; and
the fourth amplicon is a PCR product of about 3.4 kb in length.

6. The method of claim 1 wherein
the first and second primers flank the CYP21A gene if present; and
the third and fourth primers flank the CYP21A2 gene if present;
the third and second primers flank the CYP21A2/CYP21A rearranged gene if present; and
the first and fourth primers flank the CYP21A/CYP21A2 fusion gene if present.

7. The method of claim 1 wherein one or more of said first, second, third, or fourth primers are selected from the group consisting of:
SEQ ID NO 1 TCCCCAATCCTTACTTTTTGTC;:
SEQ ID NO 2 CCTCAATCCTCTGCGGCA;:
SEQ ID NO 3 GCTTCTTGATGGGTGATCAAT;:
and
SEQ ID NO 4 CCTCAATCCTCTGCAGCG;:
or complementary sequences thereof.

8. The method of claim 7 wherein
the first primer has the sequence SEQ ID NO 1: TCCCCAATCCTTACTTTTTGTC;
the second primer has the sequence SEQ ID NO 2: CCTCAATCCTCTGCGGCA;
the third primer has the sequence SEQ ID NO 3: GCTTCTTGATGGGTGATCAAT; and
the fourth primer has the sequence SEQ ID NO 4: CCTCAATCCTCTGCAGCG.

9. The method of claim 1 wherein the method further comprises separation of the amplicon(s) produced by agarose gel electrophoresis.

10. The method of claim 1 wherein the method further comprises separation of the extended extension primer(s) by capillary electrophoresis.

11. The method of claim 1, further comprising amplifying said DNA sample using two or more additional amplification primers selected to amplify one or more control nucleic acid sequences.

12. The method of claim 1, wherein the extension primers used include all of SEQ ID NOs: 5-17.

13. A method of diagnosing congenital adrenal hyperlasia in a patient, comprising:
(a) amplifying the DNA using four amplification primers to produce
(i) a first amplicon corresponding to a full length CYP21A gene if present, utilizing a first primer and a second primer selected to produce the first amplicon in the presence of the full length CYP21A gene, but not in absence of the full length CYP21A gene,
(ii) a second amplicon corresponding to a full length CYP21A2 gene if present, utilizing a third primer and a fourth primer selected to produce the second amplicon in the presence of the full length CYP21A2 gene, but not in the absence of the full length CYP21A2 gene,
(iii) a third amplicon corresponding to a CYP21A/CYP21A2 fusion gene if present utilizing one of said first primer and second primer and one of said third primer and fourth primer to produce the third amplicon in the presence of the CYP21A/CYP21A2 fusion gene, but not in absence of the CYP21A/CYP21A2 fusion gene, and
(iv) a fourth amplicon corresponding to a CYP21A2/CYP21A rearranged gene if present utilizing one of said first primer and second primer and one of said third primer and fourth primer to produce the fourth amplicon in the presence of the CYP21A2/CYP21A rearranged gene, but not in absence of the CYP21A2/CYP21A rearranged gene;
(b) determining which of the first, second, third, and fourth amplicons are produced;
(c) determining the genotype in the amplicons by using two or more oligonucleotides selected from SEQ ID NOs 5-17 or from SEQ ID Nos 18-32.

14. The method of claim 13 wherein step (c) use of SEQ ID NOs 5-17 is achieved by primer extension with distinctively labeled ddNTP from a set of ddNTPs.

15. The method of claim 13 wherein the ddNTPs comprise a fluorescent label.

16. An aqueous solution comprising one or more nucleic acids having sequences selected from the group consisting of SEQ ID NO: 18-32 or a complementary or conservative nucleic acid sequence thereof.

17. The aqueous solution of claim 16 having sequences selected from the group consisting of SEQ ID NO: 18-32.

18. An aqueous solution comprising two or more of the nucleic acids having sequences selected from the group consisting of SEQ ID NOs: 5-17 or a complementary or conservative nucleic acid sequence thereof.

19. The aqueous solution of claim 18 having two or more sequences selected from the group consisting of SEQ ID NO: 5-17.

20. A kit comprising one or more nucleic acids selected from the group consisting of SEQ ID NOs 18-32 or complementary or conservative nucleic acid sequences thereof, in an amount sufficient to perform cycle sequencing of a nucleic acid sample.

21. The kit of claim 20 wherein the one or more nucleic acids are each of SEQ ID NOs: 18-32.

22. A kit comprising two or more nucleic acids selected from the group consisting of SEQ ID NOs 5-17 or complementary or conservative nucleic acid sequences thereof, in an amount sufficient to perform a single base extension reaction.

23. The kit of claim 22 further comprising reagents for performing a single base extension reaction, wherein the nucleic acids and reagents are provided in a container.

24. The kit of claim 22 comprising each of the nucleic acids selected from the group consisting of SEQ ID NO: 15-17.

25. A kit comprising one or more nucleic acids selected from the group consisting of SEQ ID NOs 18-32 or complementary or conservative nucleic acid sequences thereof, in an amount sufficient to perform cycle sequencing of a nucleic acid sample.

26. A kit comprising one or more of more nucleic acids selected from the group consisting of SEQ ID NO: 1-4 and two or more nucleic acids selected from the group consisting of SEQ ID NOs 5-17 or complementary or conservative nucleic acid sequences thereof, in an amount sufficient to perform a single base extension reaction.

27. A kit comprising one or more nucleic acids selected from the group consisting of SEQ ID NO: 1-4 and one or more of SEQ ID NOs 18-32 or complementary or conservative nucleic acid sequences thereof in an amount sufficient to perform cycle sequencing of a nucleic acid sample.
